(19) United States
(12) Patent Application Publication
(10) Pub. No.: US 2008/0183044 A1
(43) Pub. Date: Jul. 31, 2008

(54) FLEXIBLE SURGICAL RETRACTOR AND METHOD OF USE

(76) Inventors: Dennis Colleran, North Attlebor, MA (US); Stefan Gabriel, Mattapoisett, MA (US); Noelle Dye, Charlestown, MA (US)

Correspondence Address: CARR LLP (IST), 670 FOUNDERS SQUARE, 900 JACKSON STREET, DALLAS, TX, 75202, US

(21) Appl. No.: 12/021,119

(22) Filed: Jan. 28, 2008

Related U.S. Application Data
(60) Provisional application No. 60/886,704, filed on Jan. 26, 2007.

(51) Int. Cl. A61B 1/32

(52) U.S. Cl. 600/208

(57) ABSTRACT

Disclosed herein are various embodiments that related to a surgical retractor. In certain embodiments, the systems may include a laterally expandable sleeve, sleeve activators, and insertion tools. In other embodiments, there is disclosed various methods of retraction during surgery. In some embodiments, the methods include expanding a sleeve using a sleeve activator. In some embodiments described herein, a kit may be provided for a surgical procedure. The kit may include a flexible retractor and a retractor inserter.

### PRIORITY CLAIM AND RELATED APPLICATIONS

This application claims priority to U.S. Provisional Patent Application 60/886,704 entitled “SURGICAL RETRACTOR DEVICE AND METHOD OF USE” to Spitler et al. filed Jan. 26, 2007, which is incorporated by reference in its entirety. This application also incorporates by reference U.S. Utility application Ser. No. 11/689,881, entitled “SURGICAL RETRACTOR DEVICE AND METHOD OF USE” to Colleran, filed on Mar. 22, 2007.

### TECHNICAL FIELD

The present invention relates generally to surgical retractors. More particularly, the invention relates to a flexible surgical retractor for minimally invasive procedures.

### BACKGROUND INFORMATION

The human spine provides a vast array of functions, many of which are mechanical in nature. The spine is constructed to allow nerves from the brain to pass to various portions of the middle and lower body. These nerves, typically called the spinal cord, are located in a region within the spine called the spinal canal. Various nerve bundles emerge from the spine at different locations along the lateral length of the spine. In a healthy spine, these nerves are protected from damage and/or undue pressure thereon by the structure of the spine itself.

The spine has a complex curvature made up of a plurality of individual vertebrae (typically twenty-four) separated by intervertebral discs. The intervertebral discs hold the vertebrae together in a flexible manner so as to allow relative movement between the vertebrae from front to back and from side to side. This movement allows the body to bend forward and backward, to bend from side to side, and to rotate about a vertical axis. When the spine is operating properly, the nerves are maintained clear of the hard structure of the spine throughout the available ranges of motion.

Over time or because of accidents or disease, the intervertebral discs may lose height or become cracked, dehydrated, or herniated. The result is that the height of one or more discs may be reduced. The reduction in height can lead to compression of the nerve bundles. Such compression may cause pain and, in some cases, damage to the nerves.

Currently, there are many systems and methods at the disposal of a physician for reducing or eliminating the pain by minimizing the stress on the nerve bundles. In some instances, the existing disc is removed and an artificial disc is substituted therefore. In other instances, two or more vertebrae are fused together to prevent relative movement between the fused discs.

In some procedures, minimally invasive surgical procedures have been developed to fuse or otherwise treat vertebrae. Such procedures can reduce pain, post-operative recovery time, and the destruction of healthy tissue. Minimally invasive surgical procedures are particularly desirable for spinal and neurosurgical applications because of the need for access to locations deep within the body and the possible range of damage to vital intervening tissues.

Generally, it is desirable to access the surgical site using minimally invasive techniques or portals, rather than through a significant incision, to aid in preserving the integrity of the intervening tissues. In such procedures, however, it may be necessary to hold the edges of an incision apart to provide a clear operating field within which the surgeon can operate.

What is needed, therefore, is a tool or retractor adapted to work with minimally invasive procedures that allows the surgeon to have a clear path to the operating field, and a method for using such a tool or retractor.

### SUMMARY

Disclosed herein are various embodiments that related to systems incorporating a surgical retractor. In certain embodiments, the systems may include a laterally expandable sleeve, sleeve activators, and insertion tools. In other embodiments, there is disclosed various methods of retraction during surgery. In some embodiments, the methods include expanding a sleeve using a sleeve activator.

In some of the disclosed embodiments, a kit may be provided for a surgical procedure. The kit may include a flexible retractor and a retractor inserter. In certain embodiments, the kit may also include at least one illumination source configured to couple to the flexible retractor. In some embodiments, the kit may include a sequential dilator set.

### BRIEF DESCRIPTION OF THE DRAWINGS

These and other features, and advantages, will be more clearly understood from the following detailed description taken in conjunction with the accompanying drawings. It is important to note the drawings are not intended to represent the only aspect of the invention.

FIG. 1a is a front view of one embodiment of a surgical retractor which incorporates one or more aspects of the present invention.

FIG. 1b is the retractor of FIG. 1a in an expanded or second configuration.

FIG. 2 is a side view of a retractor inserter.

FIG. 3 is an embodiment of the surgical retractor of FIG. 1 coupled to a retractor frame.

FIG. 4 is an embodiment of tray used in a surgical kit incorporating one or more aspects of the present invention,

FIGS. 5a to 5d illustrate one embodiment of the use of the retractor of FIG. 1.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

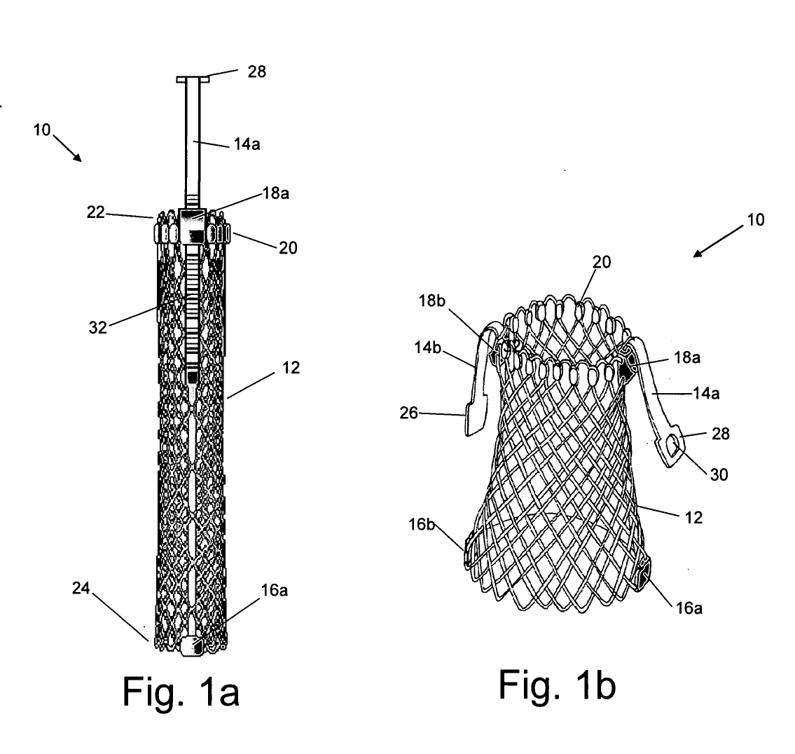

FIG. 1a depicts an embodiment of a surgical retractor 10 in a first or laterally collapsed configuration. In contrast, FIG. 1b depicts the surgical retractor 10 in a second or laterally expanded condition. In some embodiments, the surgical retractor 10 comprises an expandable sleeve 12, sleeve expansion activators or side straps 14a and 14b, strap anchors 16a and 16b, strap locks 18a and 18b. In certain embodiments, there also may be a plurality of mesh pads 20.

The expandable sleeve 12 has a proximal end portion 22 and a distal end portion 24. In some embodiments, the expandable sleeve 12 may be a tubular structure formed of a flexible material, such as mesh, wire, or polymer material. For purposes of convenience, the term “mesh” is used throughout the disclosure to refer to any such flexible material. The mesh may be selected to provide a desired level of rigidity while still being deformable within certain parameters. For example, the mesh may have a level of rigidity that requires application of a certain amount of force in order for the mesh to be expanded or collapsed. Accordingly, when expanded, the mesh may be rigid enough to resist collapsing under the pressure exerted by the surrounding tissue, while still maintaining a level of malleability that allows the mesh to be expanded/collapsed when acted upon by external forces different from and/or larger than the pressure exerted by the surrounding tissue. In some embodiments, the mesh may be made from a shape memory alloy that reacts to the application of heat, electricity, or other means to enable the retractor to take on its original shape to ease removal from the body.

In certain embodiments, the mesh may allow for a better distribution of forces on the tissue than a similarly shaped rigid wall, and may thereby decrease stress and potential damage to the tissue during retraction.

The mesh portion of the wall may be formed using various mesh patterns and thicknesses and may be formed to have a desired level of malleability. In certain embodiments, the mesh may be made of braided wire to form the expandable sleeve 12. In certain embodiments, such as illustrated in FIGS. 1a and 1b, the sleeve 12 may utilize a biaxial braid in an over 1 under 1 diamond pattern. A biaxial braid is a form of weaving that causes the tubular form to be very compliant and to radially expand when the tube is longitudinally shortened and to radially contract when the tube is stretched longitudinally. Other weave patterns and embodiments of the mesh material are possible and may include different patterns and/or materials.

In certain embodiments, individual strands may be encased in a polymer, elastomer or other protective material. The wire frame may also be covered by a sheet of flexible elastomer (e.g., the mesh portion may be placed between two sheets of polymer or elastomer or the mesh portion may be molded within) to protect the tissue during use. In some embodiments, the ends of the tubular structure may also include a protective leading edge made, for example, of polymer/elastomer having an increased thickness. In other embodiments, the end or ends of the tubular structure may include the plurality of mesh pads 20.

In some embodiments, there may be one or more sleeve expansion activators, such as the side straps 14a and 14b. In the illustrative embodiment, the side straps 14a and 14b may be coupled to the distal end of the sleeve using strap anchors 16a and 16b. In certain embodiments, the strap anchors 16a-16b may be enlarged formed plastic couplers integral with the side straps. In certain embodiments, the strap anchors 16a-16b may be formed or pressed fitted over the turns of two or more wire strands. In some embodiments, the strap anchors 16a-16b may have interior side projections and/or grooves which engage insertion instrumentation.

In some embodiments, the proximal ends of the side straps 14a and 14b may have couplers 26 and 28. For instance, coupler 26 may be an enlarged end having a side projection (not shown). Coupler 28 may be an enlarged end with an eye portion 30 sized to receive the side projection. Once the side projection is received by the eye portion, the couplers 26 and 28 may be snapped in place to join the proximal ends of the respective side straps.

In certain embodiments, the side straps 14a-14b may have a series of transverse serrations 32 on one or both faces of the respective strap. In such embodiments, the overall cross-sectional profile of the side straps 14a-14b may be increased to provide the same tensile strength when the cross-sectional profile and strap strength is a reduced due to the serrations 32.

The strap locks or ratchet locks 18a-18b may include an opening or channel sized to accommodate the cross-sectional shape of the side straps 14a-14b. The strap locks 18a-18b may have a series of engagement teeth or pawls (not shown) shaped to cooperatively engage one or more of the transverse serrations of the side straps 14a and 14b. The teeth are shaped such as to permit passage of the straps in one direction and to prevent subsequent withdrawal of the straps in an opposite direction.

In some embodiments, the strap locks 18a-18b may also include a release lever coupled to the engagement teeth. When the release lever is pivoted in one direction, it pivots the engagement teeth away from the serrations 32 so the side straps 14a-14b may freely move with respect to the strap locks. One example of a locking system may be found in U.S. Pat. No. 3,924,299 which is incorporated by reference. In certain embodiments, the strap locks 18a-18b may be shaped so that they can be easily engaged and disengaged with a finger.

Because the strap locks are coupled to the proximal portion of the sleeve 12, moving the strap locks 18a-18b from a proximal position illustrated in FIG. 1a towards a distal position illustrated in FIG. 1b, shortens the overall length of the retractor 10. As noted previously, a weave configuration of the sleeve 12 is used so that when the length of the retractor is decreased, the lateral diameter of the retractor is increased which causes the retractor to expand laterally. Once expanded, the strap locks hold the mesh in place until released by the user.

In certain embodiments, the side straps 14a-14b may be formed from a translucent material, such as certain forms of plastic. In other embodiments, it is understood that the material may have additional properties, such as being capable of withstanding a high heat sterilization procedure such as autoclaving. The translucent material may enable light to shine through the retractor to illuminate the interior of the sleeve and the corresponding surgical area.

In such embodiments, at the proximal end portions of the side straps 14a-14b, there may be one or two slots and for coupling to a light coupler (not shown). In some embodiments, a first end of an optical cable may be coupled to a light source, and a second end of the optical cable may be coupled to the side straps 14a-14b. In certain embodiments, the straps may have may have one or more angled cuts at the proximal end to specifically direct light to particular regions of the operating field.

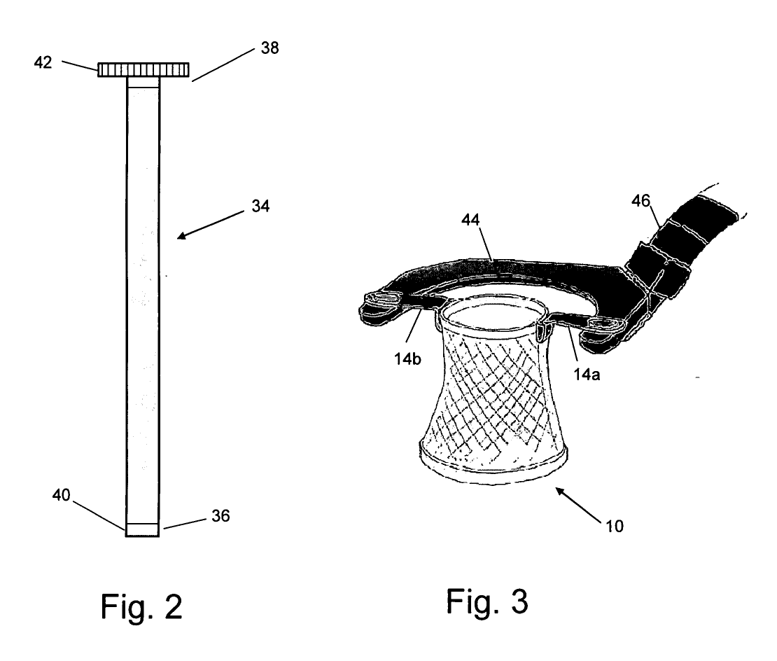

Turning now to FIG. 2, there is a side view of one embodiment of an insertion instrument or retractor inserter 34. The insertion instrument 34 has a distal end portion 36 and a proximal end portion 38. In certain embodiments, there may be a lateral lip 40 at the distal end portion 36 which is sized to slidingly engage and disengage corresponding recesses and/or projections on the interior faces of strap anchors 16a-16b (not shown). At the proximal end portion 38 of the insertion instrument 34, there may be a turning knob 42. In certain embodiments, the longitudinal length of the insertion instrument is sized so that the instrument can fit within the loop created when the side straps 14a and 14b are joined using couplers 26 and 28 as shown in FIG. 1a. In other embodiments, the instrument may have a traditional longitudinal handle.

As illustrated in FIG. 3, once expanded, the surgical retractor 10 may couple to a retractor frame 44. The use of the retractor frame 44 allows the surgical retractor 10 to be coupled to a surgical table to stabilize and fix the position of the surgical retractor relative to the patient. In the illustrated embodiment of FIG. 3, the side straps 14a-14b couple the surgical retractor to the retractor frame 44. In other embodiments, other portions of the surgical retractor may include connection features that allow the surgical retractor to be coupled to the surgical table such as, but not limited to recesses, threaded openings, protrusions, grooves, slots, and/or quick release mechanisms. In certain embodiments, the retractor frame 44 may be coupled to a flexible arm 46, such as MediFlex Arm and Table Mount (from Mediflex Surgical Products, Islandia, N.Y.).

The surgical retractor 10 may be provided as part of a surgical kit. The kit may include one or more cases that hold accessories, instruments, and retractors. The cases may have a plurality of openings. In certain embodiments, the entire case may be placed in a sterilizer to sterilize all of the contents within the case. Some of the contents in the case may be pre-sterilized and placed in bags that are put into the case. Other components of the kit may include a table adaptor, light cables and adaptors, disposable light mats, and trays.

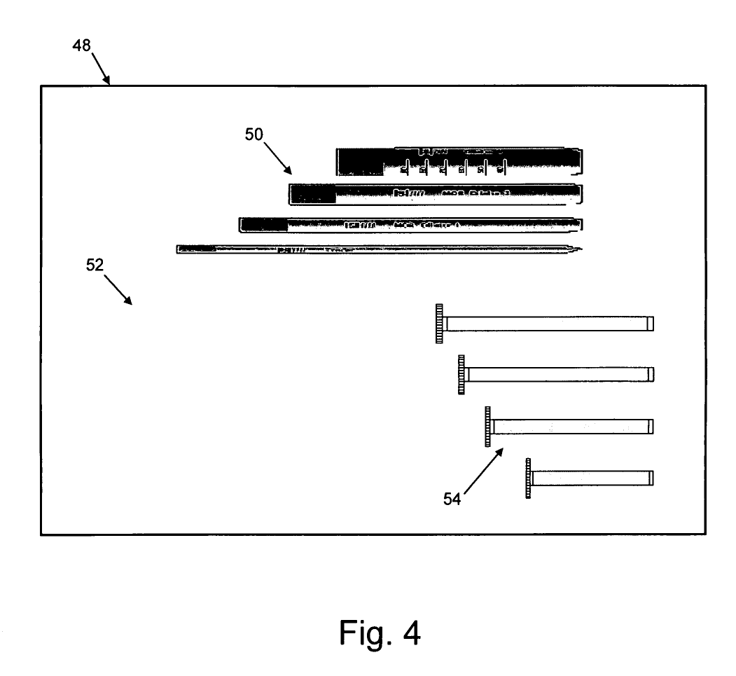

One embodiment of a tray 48 of a kit is illustrated in FIG. 4. The instruments included in the tray 48 may include a dilator set 50, a set of sleeve retractors 52, and a set of retractor inserters 54. In certain embodiments, the dilator set 50 may be used to expand the initial incision made in the patient. The set of retractors 52 could have different lengths depending on the anatomy and the procedure. For instance, in transforaminal or posterior lumbar approaches, the retractor lengths included in the kit may have lengths corresponding to incision lengths of 40 mm, 50 mm, 60 mm, 70 mm, and 80 mm. For extreme lateral lumbar approaches (e.g., an XLIF procedure), the retractor lengths included in the kit may have lengths corresponding to incision of 90 mm, 100 mm, 110 mm, 120 mm, and 130 mm. Other sizes and quantities may also be included in the kit. In certain embodiments, the set of retractor inserters 54 may correspond to the retractor lengths.

In certain embodiments, there may also be one or more retractor frames 44 (FIG. 3) included in the kit. A number of different retractor frames may be provided where each retractor frame has different size interior shape or diameter. Different sized frames will allow for various sized openings and clearances for different procedures. Surgeons may have different preferences on the size of the openings or the angle. So, providing a plurality of retractor frames allow the surgeon to determine the best configuration for the surgery.

Various surgical accessories in the retractor kit may be prepared before the surgery. For instance, a flexible arm may be attached to the surgery table or to a table mount. If translucent side strips are used, the light source may be prepared and optical cables inserted. A retractor frame may be selected and coupled to the flexible arm or a table mount.

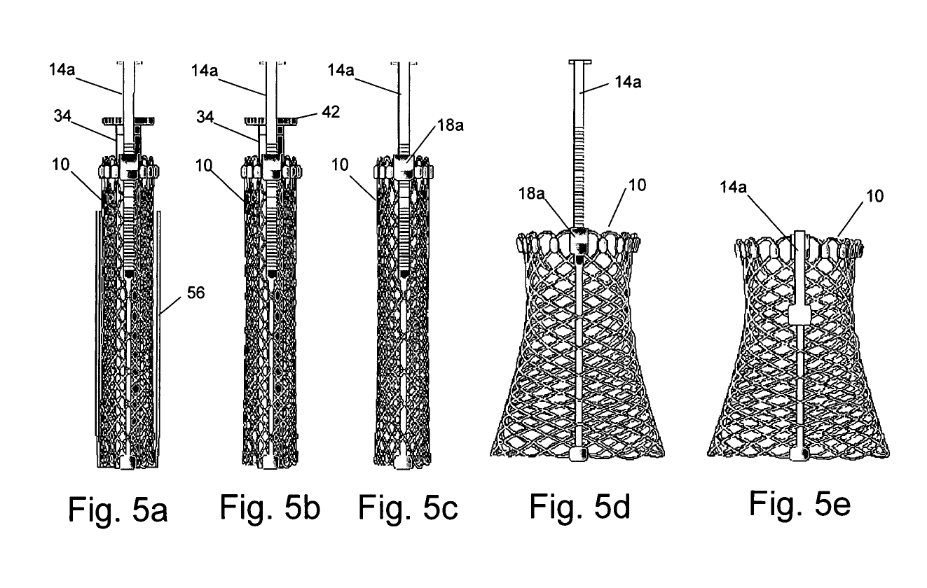

When the surgical procedure begins, an incision may be formed in the patient. The incision may be expanded using the dilator set 50 (FIG. 4). Once expanded the smaller dilators may be removed, leaving the largest dilator in the incision (e.g. a 22 mm dilator). A sleeve retractor of a particular length may then be selected and coupled to a retractor inserter. As illustrated in FIG. 5a, the retractor inserter 34 and the retractor 10 may then be inserted into the dilator 56. Note that in FIG. 5a, the side straps 14a and 14b are coupled together and only strap 14a is visible.

FIG. 5b illustrates the situation where the dilator 56 has been removed from the incision. At this point the retractor inserter 34 may be disconnected from the retractor 10 by turning the retractor inserter 34 with the turning knob 42. The retractor inserter 34 can then be removed as illustrated in FIG. 5c.

FIG. 5d illustrates the situation where the sleeve has been expanded by a surgeon pushing down on the strap locks 18a-18b while pulling up on the side straps 14a and 14b. The side straps 14a-14b may then be decoupled from each other and pulled down to the sides (FIG. 5e). In certain embodiments, the side straps 14a-14b may be adapted to couple to a retractor frame as illustrated in FIG. 3.

The retractor and retractor frame may then be positioned according to the situation. Correct positioning may require checking the retractor angulation with respect to the patient. Furthermore, positioning of the retractor and flexible arm may need to be checked for proper optical and radiographic visualization relative to the patient's anatomy. The table mount and/or flexible arm may then be locked in position. In certain embodiments, optic cables may be coupled to optical couplings in the straps.

After the surgical retractor is positioned and set up, the surgical procedure may be performed. After the surgical procedure, the side straps 14a-14b may be released—which will collapse the retractor. The surgical retractor may then be removed from the patient.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

It is understood that terms such as “side”, “top”, “bottom”, “front”, “back”, “proximal”, and “distal” are relative and may be interchangeable depending on the perspective from which the device of the present disclosure is being viewed. Accordingly, such terms are used for purposes of illustrating and describing various embodiments of the present disclosure and are not intended to be limiting. When the term “proximal” is used it refers to the portion of a component that is closer to the user when the embodiment is used in its intended manner. Similarly, when the term “distal” is used, the term refers to the portion of a component that is farther from the user when the embodiment is used in its intended manner.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

Any advantages and benefits described may not apply to all embodiments of the invention. When the word “means” is recited in a claim element, Applicant intends for the claim element to fall under 35 USC 112, paragraph 6. Often a label of one or more words precedes the word “means”. The word or words preceding the word “means” is a label intended to ease referencing of claims elements and is not intended to convey a structural limitation. Such means-plus-function claims are intended to cover not only the structures described herein for performing the function and their structural equivalents, but also equivalent structures. For example, although a nail and a screw have different structures, they are equivalent structures since they both perform the function of fastening. Claims that do not use the word means are not intended to fall under 35 USC 112, paragraph 6.

The foregoing description of the embodiments of the invention has been presented for the purposes of illustration and description. It is not intended to be exhaustive or to limit the invention to the precise form disclosed. Many combinations, modifications and variations are possible in light of the above teaching. Undescribed embodiments which have interchanged components are still within the scope of the present invention. It is intended that the scope of the invention be limited not by this detailed description, but rather by the claims appended hereto.

What is claimed is:

1. A surgical retractor, comprising:
a braided mesh sleeve having a proximal end portion and a distal end portion,
a first strap anchor coupled to the distal end portion,
a second strap anchor coupled to the distal end portion,
a first sliding lock coupled to the proximal end portion,
a second sliding lock coupled to the proximal end portion,
a first strap coupled to the first strap anchor and extending through the first sliding lock,
a second strap coupled to the second strap anchor and extending through the second sliding lock,
wherein the mesh sleeve is adapted to be expanded from a first position to a second position when the first and second sliding locks are each moved longitudinally towards the first and second strap anchors, respectively.

2. The surgical retractor of claim 1 wherein the braided mesh sleeve is made using a biaxial braid.

3. The surgical retractor of claim 1 wherein the braided mesh sleeve is made using a biaxial braid in an over 1 under 1 diamond braid pattern.

4. The surgical retractor of claim 1 wherein the mesh is made from the group consisting of at least one wire and a polymer material.

5. The surgical retractor of claim 1 wherein portions of the braided mesh are at least partially encased in a flexible shell.

6. The surgical retractor of claim 1, wherein the mesh is formed using a shape memory alloy.

7. The surgical retractor of claim 1, wherein the first strap comprises a plurality of transverse serrations on one side of the first strap.

8. The surgical retractor of claim 7, wherein the first sliding lock further comprises:
an opening sized to accommodate a cross-sectional shape of the first strap,
at least one tooth shaped to cooperatively engage at least one of the transverse serrations such as to permit passage of the first strap in one direction and to prevent subsequent withdrawal of the strap in an opposite direction.

9. The surgical retractor of claim 8, wherein the first sliding lock further comprises a release lever coupled to the at least one tooth to disengage the at least one tooth from the plurality of transverse serrations so as to permit passage of the first strap.

10. The surgical retractor of claim 1 wherein the first strap is composed of a translucent material and includes light receiving couplers formed in a proximal portion of the first strap.

11. A surgical kit, comprising:
a plurality of retractors adapted to expand from a lateral unexpanded condition to a lateral expanded condition, wherein each retractor in the plurality of retractors includes:
a tubular mesh having a proximal and distal end portion,
at least one lateral expansion activator coupled to a side wall of the tubular mesh,
at least one retractor inserter adapted to fit within each retractor in the lateral unexpanded condition and to couple and decouple from the respective distal end portion of each retractor,
at least one retractor frame, and
a plurality of sequential dilators.

12. The surgical kit of claim 11, wherein the at least one lateral expansion activator includes a side strap coupled to the distal end portion and slidingly coupled to the proximal end portion.

13. The surgical kit of claim 12 wherein the at least one lateral expansion activator includes a ratchet lock coupled to the proximal end portion and slidingly coupled to the side strap.

14. The surgical retractor kit of claim 11 wherein each retractor in the plurality of retractors has a different longitudinal length from the other retractors in the plurality of retractors

15. The surgical kit of claim 11, further comprising:
a mount to couple to each of the plurality of tubular mesh adapters,
a flexible arm to couple to the mount,
a table mount to couple the flexible arm to an operating table.

16. A method of using a surgical retractor comprising:
coupling a distal end of a retractor sleeve to a distal end of an insertion tool,
inserting the insertion tool and sleeve into a dilator,
removing the dilator,
decoupling the insertion tool from the sleeve and removing the insertion tool from the sleeve,
activating the sleeve to cause a lateral expansion of the sleeve to enlarge a void defined by the sleeve, and
locking the activator in place.

17. The method of claim 16 wherein the activating further comprises sliding a proximal portion of the retractor sleeve down a side strap which is coupled to a distal portion of the retractor sleeve to cause the retractor sleeve to expand laterally with respect to a longitudinal axis of the retractor sleeve.

18. The method of claim 17 wherein the activating further comprises sliding a ratchet lock coupled to the proximal portion of the retractor sleeve down a side strap which is coupled to a distal portion of the retractor sleeve to cause the retractor sleeve to expand laterally with respect to a longitudinal axis of the retractor sleeve.

19. The method of claim 18 wherein the locking comprises engaging at least one tooth in the ratchet lock to a corresponding serration in the side strap.

20. The method of claim 15, further comprising:
forming an incision in a tissue at a surgical site;
sequentially dilating the tissue surrounding the surgical site to form a sequentially dilated surgical opening; and
inserting the retractor sleeve into the sequentially dilated surgical opening.
